(19) United States
(12) Patent Application Publication
(10) Pub. No.: US 2020/0090802 A1
(43) Pub. Date: Mar. 19, 2020

(54) Systems and Methods for Automated Treatment Recommendation Based on Pathophenotype Identification

(71) Applicant: The Brigham and Women's Hospital, Inc., Boston, MA (US)

(72) Inventor: Bradley A. Maron, Cambridge, MA (US)

(21) Appl. No.: 16/496,932

(22) PCT Filed: Mar. 23, 2018
(86) PCT No.: PCT/US18/24152

§ 371 Date: Sep. 23, 2019

Related U.S. Application Data
(60) Provisional application No. 62/475,955, filed on Mar. 24, 2017.
(60) Provisional application No. 62/624,300, filed on Jan. 31, 2018.

(51) Int. Cl. G16H 20/30; G16H 50/30

(52) U.S. Cl. CPC G16H 20/30; G16H 50/30

(57) ABSTRACT

A system and method are provided automated treatment recommendations based on pathophenotype identification. The computer system may receive variable values corresponding to a subject to which a clinical test has been applied and may normalize and vectorize these variables and may compare the resultant vector to centroids of predefined vector clusters. The computer system may assign the subject to a cohort corresponding to the vector cluster having a centroid with the shortest Euclidean distance to the vector. Based on this cohort, the computer system may generate and display prognostic information which may include any of: a recommended course of treatment, a pathophenotype corresponding to the cohort, risk of hospitalization of the subject, and an alert recommending hospitalization of the subject.

### CROSS-REFERENCE TO RELATED APPLICATIONS

This application is based on, claims priority to, and incorporates herein by reference in its entirety U.S. Provisional Application 62/475,955, filed Mar. 24, 2017, and U.S. Provisional Application 62/624,300, filed Jan. 31, 2018.

### STATEMENT REGARDING FEDERALLY SPONSORED RESEARCH

This invention was made with government support under 1K08HL11207-01A1, 1R56HL131787-01A1, 1 K08HL128802-01A1, U01 HL125215, HL061795, HG007690, HL108630, and GM107618 awarded by the National Institutes of Health. The government has certain rights in the invention.

### BACKGROUND

Exercise intolerance is highly prevalent across a wide range of diseases encountered commonly in routine clinical practice, and is a principal cause of morbidity and increased healthcare cost burden. The pathophysiology of exercise intolerance is generally ascribed to a cardiovascular, pulmonary, or skeletal muscle abnormality that impairs oxygen (O₂) delivery to or extraction by peripheral tissue. However, abnormalities in multiple organ systems are frequently observed in patients referred for evaluation, complicating efforts to establish the parameters that delineate different forms of exercise intolerance as well as the development of patient-specific risk stratification metrics. This dilemma is due, in part, to conventional methods that utilize only a narrow subset of available clinical data for analyzing exercise performance. As a result, peak volume of oxygen consumption (pVO₂) is often used as the single exercise variable for determining prognosis in patients with cardiopulmonary diseases. Interpreting exercise data using a wider range of clinical variables may have important implications for understanding exercise subtypes and clarifying patient prognosis, but such methods are not currently available.

It is within this context that embodiments of the present invention arise.

### SUMMARY OF THE DISCLOSURE

The present disclosure provides systems and methods for automated risk stratification and generation of treatment recommendation for medical conditions and diseases. As will be described, these systems and methods provide greater flexibility and improved results compared to conventional risk stratification and treatment recommendation methods.

In an embodiment, a method may include steps for receiving, with a computer system, values of one or more variables corresponding to a subject to which a diagnostic test has been administered; with a processor of the computer system, generating at least one vector from the values of the one or more variables; with the processor, determining a plurality of Euclidean distances between the at least one vector and respective centroids of each of a plurality of predefined vector clusters corresponding to a plurality of pathophenotypes; with the processor, identifying a pathophenotype corresponding to the predefined vector cluster of the plurality of predefined vector clusters corresponding to the shortest Euclidean distance of the plurality of Euclidean distances; with the processor, assigning the subject to a cohort based on the identified pathophenotype; with the processor, determining a recommended course of treatment based on the cohort to which the subject has been assigned; and presenting the recommended course of treatment on an electronic display of the computer system.

In some embodiments, the diagnostic test may include invasive cardiopulmonary exercise testing (iCPET).

In some embodiments, the one or more variables may include at least one of, but are not limited to: peak minute ventilation, forced expiratory volume in one second, peak stroke volume, maximum voluntary ventilation, forced vital capacity, peak arterial to mixed venous oxygen content difference, peak arterial pH, peak arterial lactate, peak arterial oxygen content, and peak rate of oxygen consumption.

In some embodiments, the recommended course of treatment may include initiation of pharmacotherapeutic intervention including treatment with a predetermined class of cardiovascular drugs that can be classified to ten categories based on the features, including, but not limited to pulmonary vasodilator therapy, pulmonary arterial hypertension treatment, peripheral vasodilator reaming enhancer, angiotensin converting agent inhibitor, hypotension and shock therapeutic agent, diuretic, antiarrhythmic agent, antiarralciton drug, antihypertensive agent, anticoagulant and thrombolytic agent, and cardiac tonic therapeutic agents including, but not limited to beta blockers and calcium channel blockers.

In some embodiments, the recommended course of treatment may include optimization of bronchodilator therapy, inhaled corticosteroid therapy, muscarinic agents, or immunomodulating agents.

In some embodiments, the method may further include, with the processor, automatically generating an alert based on the cohort to which the subject has been assigned, wherein the alert recommends outcomes including, but not limited to immediate hospitalization of the subject, risk of mortality of the subject, pharmacotherapeutic initiation, and pharmacotherapeutic escalation; and presenting the alert on the electronic display.

In some embodiments, the plurality of predefined vector clusters comprises additional values for each of the one or more variables. The method may further include, with the processor, normalizing each of the values of the one or more variables corresponding to the subject relative to a respective mean and a respective variance of corresponding values of the additional values to have an updated mean of zero and an updated variance of one before generating the at least one vector.

In an embodiment, a method may include administering, with an invasive cardiopulmonary exercise testing (iCPET) system, an iCPET test to a subject; during the administration of the iCPET test, continuously collecting and storing iCPET data captured in real-time by the iCPET system; with a computer processor, analyzing values of one or more variables of the iCPET data using network analysis to identify an exercise pathophenotype of the subject; with the computer processor, assigning the subject to a cohort based on the identified exercise pathophenotype; with the computer processor, determining a recommended course of treatment based on the cohort to which the subject has been assigned; and presenting the recommended course of treatment on an electronic display.

In some embodiments, analyzing the values of the one or more variables of the iCPET data using network analysis to identify an exercise pathophenotype of the subject further includes, with the computer processor, generating at least one vector from the values of the one or more variables; with the computer processor, determining a plurality of Euclidean distances between the at least one vector and respective centroids of each of a plurality of predefined vector clusters corresponding to a plurality of exercise pathophenotypes; and with the computer processor, identifying the exercise pathophenotype as that which corresponds to the predefined vector cluster of the plurality of predefined vector clusters corresponding to the shortest Euclidean distance of the plurality of Euclidean distances.

In some embodiments, the one or more variables may include at least one of, but are not limited to: peak minute ventilation, forced expiratory volume in one second, peak stroke volume, maximum voluntary ventilation, forced vital capacity, peak arterial to mixed venous oxygen content difference, peak arterial pH, peak arterial lactate, peak arterial oxygen content, and peak rate of oxygen consumption.

In some embodiments, the one or more variables may peak minute ventilation, forced expiratory volume in one second, peak stroke volume, maximum voluntary ventilation, forced vital capacity, peak arterial to mixed venous oxygen content difference, peak arterial pH, peak arterial lactate, peak arterial oxygen content, and peak rate of oxygen consumption.

In some embodiments, the recommended course of treatment may include initiation of pharmacotherapeutic intervention including treatment with a predetermined class of cardiovascular drugs that can be classified to ten categories based on the features, including, but not limited to pulmonary vasodilator therapy, pulmonary arterial hypertension treatment, peripheral vasodilator reaming enhancer, angiotensin converting agent inhibitor, hypotension and shock therapeutic agent, diuretic, antiarrhythmic agent, antiarralciton drug, antihypertensive agent, anticoagulant and thrombolytic agent, and cardiac tonic therapeutic agents including, but not limited to beta blockers and calcium channel blockers.

In some embodiments, the recommended course of treatment may include optimization of bronchodilator therapy, inhaled corticosteroid therapy, muscarinic agents, or immunomodulating agents.

In some embodiments, the method may further include, with the computer processor, automatically generating an alert based on the cohort to which the subject has been assigned, wherein the alert recommends immediate hospitalization of the subject; and presenting the alert on the electronic display.

In some embodiments, analyzing the values of the one or more variables of the iCPET data using network analysis to identify an exercise pathophenotype of the subject may further include, with the computer processor, normalizing each of the values of the one or more variables to have an updated mean of zero and an updated variance of one relative to a respective mean and a respective variance of additional values for a corresponding variable of the one or more variables represented in the plurality of predefined vector clusters; with the computer processor, generating a vector that includes the normalized values; with the computer processor, determining a plurality of Euclidean distances between the vector and respective centroids of each of a plurality of predefined vector clusters; and with the computer processor, identifying the exercise pathophenotype as that which corresponds to the predefined vector cluster of the plurality of predefined vector clusters corresponding to the shortest Euclidean distance of the plurality of Euclidean distances.

In an embodiment, a system may include an invasive cardiopulmonary exercise testing (iCPET) system that administers an iCPET study on a subject and that generates values for a plurality of variables for the subject during the administration of the iCPET study; and a computer system that is communicatively coupled to the iCPET system. The computer system may include a memory; an electronic display; and a processor that executes instructions stored in the memory for: receiving, from the iCPET system, the values for the plurality of variables; analyzing the values of the plurality of variables using network analysis to identify an exercise pathophenotype of the subject; assigning the subject to a cohort based on the identified exercise pathophenotype; determining a recommended course of treatment based on the cohort to which the subject has been assigned; and presenting the recommended course of treatment on the electronic display.

In some embodiments, the plurality of variables may include at least one of, but is not limited to: peak minute ventilation, forced expiratory volume in one second, peak stroke volume, maximum voluntary ventilation, forced vital capacity, peak arterial to mixed venous oxygen content difference, peak arterial pH, peak arterial lactate, peak arterial oxygen content, and peak rate of oxygen consumption.

In some embodiments, each of the plurality of variables may be correlated with at least one other variable of the plurality of variables with a correlation coefficient having a magnitude greater than 0.5 and a calculated probability of less than 10⁻⁴⁰.

In some embodiments, the recommended course of treatment may include initiation of pharmacotherapeutic intervention including treatment with a predetermined class of cardiovascular drugs that can be classified to ten categories based on the features, including, but not limited to pulmonary vasodilator therapy, pulmonary arterial hypertension treatment, peripheral vasodilator reaming enhancer, angiotensin converting agent inhibitor, hypotension and shock therapeutic agent, diuretic, antiarrhythmic agent, antiarralciton drug, antihypertensive agent, anticoagulant and thrombolytic agent, and cardiac tonic therapeutic agents including, but not limited to beta blockers and calcium channel blockers.

In some embodiments, the recommended course of treatment may include initiation of pulmonary vasodilator therapy, inhaled corticosteroid therapy, muscarinic agents, or immunomodulating agents.

In some embodiments, the processor may further execute instructions for: automatically generating an alert based on the cohort to which the subject has been assigned, wherein the alert recommends immediate hospitalization of the subject; and presenting the alert on the electronic display.

In some embodiments, the processor may further execute instructions for: normalizing each of the values of the plurality of variables to have an updated mean of zero and an updated variance of one relative to a respective mean and a respective variance of additional values for a corresponding variable of the plurality of variables represented in the plurality of predefined vector clusters; generating a vector that includes the normalized values; determining a plurality of Euclidean distances between the vector and respective centroids of each of a plurality of predefined vector clusters; and identifying the exercise pathophenotype as that which corresponds to the predefined vector cluster of the plurality of predefined vector clusters corresponding to the shortest Euclidean distance of the plurality of Euclidean distances.

In an embodiment, a method may include receiving values of one or more variables corresponding to a subject to which a diagnostic test has been administered; generating at least one vector from the values of the one or more variables; determining a plurality of Euclidean distances between the at least one vector and respective centroids of each of a plurality of predefined vector clusters corresponding to a plurality of pathophenotypes; and identifying a pathophenotype corresponding to the predefined vector cluster of the plurality of predefined vector clusters corresponding to the shortest Euclidean distance of the plurality of Euclidean distances; assigning the subject to a cohort based on the identified pathophenotype.

The foregoing and other aspects and advantages of the invention will appear from the following description. In the description, reference is made to the accompanying drawings which form a part hereof, and in which there is shown by way of illustration a preferred embodiment of the invention. Such an embodiment does not necessarily represent the full scope of the invention, however, and reference is made therefore to the claims and herein for interpreting the scope of the invention.

### BRIEF DESCRIPTION OF THE DRAWINGS

FIG. 1 shows an illustrative system diagram that includes an invasive cardiopulmonary exercise testing (iCPET) system and a computer system in accordance with an embodiment.

FIG. 2 shows an illustrative flow chart depicting an approach by which a set of clinical data may be progressively refined to define a subnetwork of connected variables in accordance with an embodiment.

FIG. 3 shows an illustrative network of interconnected variables that may be generated during the application of an iCPET test in accordance with an embodiment.

FIG. 4 shows an illustrative subnetwork of interconnected variables that may be derived from the illustrative network of FIG. 3 in accordance with an embodiment.

FIG. 5 shows an illustrative chart depicting clinical data for 738 patients that were analyzed for performance on the 10 variables included in the illustrative subnetwork of FIG. 4 plotted according to variance from a first principal component and variance from a second principal component, where the patient data is divided into four clusters in accordance an embodiment.

FIG. 6 shows an illustrative chart depicting the normalized values of clinical data for each variable in the subnetwork stratified by cluster in accordance with an embodiment.

FIG. 7 shows an illustrative flow chart for a method of automatically generating and displaying prognostic information based on variables generated by administering a test to a subject in accordance with an embodiment.

### DETAILED DESCRIPTION

The systems and methods of the present invention can be utilized with a wide variety of data and systems and methods for acquiring and processing data. Some non-limiting examples of embodiments that include invasive cardiopulmonary exercise testing (iCPET) systems follow hereafter. However, the systems and methods of the present disclosure are not limited to these modalities.

As will be described, in one aspect, the present disclosure provides systems and methods for automatically risk stratifying a subject (e.g., a patient) and generating a prognosis (e.g., including treatment recommendations, risk of hospitalization, and/or alerts indicating that the subject needs to be hospitalized) based on an identified cohort to which the subject belongs. As used to herein, a “cohort” may refer to a classification of subjects with a given disease or disorder (e.g., as determined via k-means clustering), with different cohorts being associated with different pathophenotypes of the given disease or disorder (e.g., exercise dysfunction). As used herein, a “pathophenotype” of a disease or disorder refers to a particular set of observable clinical features of that disease or disorder. For example, a given disease or disorder may manifest as a spectrum of symptoms and observable clinical features with varying seventies, and different pathophenotypes may be identified in order to classify different possible expressions of the disease or disorder. The identification of different pathophenotypes as an output of the risk stratification of a subject could be applied across a number of different disorders, including but not limited to, coronary artery disease, myocardial infarction, acute coronary syndrome, sudden cardiac-death syndrome, oncology-cardiology syndromes, systemic hypertension, genetic cardiomyopathy, hypertrophic cardiomyopathy, myocarditis, pulmonary hypertension, pulmonary arterial hypertension, amyloid cardiomyopathy, solid tumor cancer, blood cancer, paraneoplastic syndromes, hematopoietic disorders, platelet disease, aortic valve disease, mitral valve disease, pulmonic valve disease, renovascular disease, cardiorenal syndrome, venothromboembolic disease, chronic obstructive pulmonary disease, asthma, interstitial lung disease, sleep apnea, chronic kidney disease, tubulointerstitial diseases of the kidney, adrenal disease, syndromes of abnormal aldosterone synthesis, thyroid disease, autoimmune disease, and diabetes. In the context of exercise dysfunction, standard pathophenotypes may include pulmonary vascular disease (PVD), left heart disease (LHD) plus PVD, LHD with no PVD, peripheral oxygen (O₂) extraction disorder, low ventricular filling syndrome (i.e., failure to augment venous return as the primary identifiable cause of impaired cardiac output), and presumed normal.

The assignment of a subject into a particular cohort may be performed based on clinical data (e.g., variable values) acquired for that subject (e.g., via the application of a clinical test such as an iCPET test). For example, the variable values corresponding to the subject may be vectorized and, optionally, normalized and may then be compared to the mean values of each cluster of vectors of multiple clusters of vectors. Each cluster of vectors may correspond to a different cohort/pathophenotype, and the vectors of the clusters of vectors (e.g., vector clusters) may each include to historical clinical data for respectively different subjects (e.g., collected during one or more initial clinical studies). The subject of the present example may then be assigned to the cohort corresponding to the cluster of vectors from which the vectorized variable values of the subject has the shortest Euclidean distance.

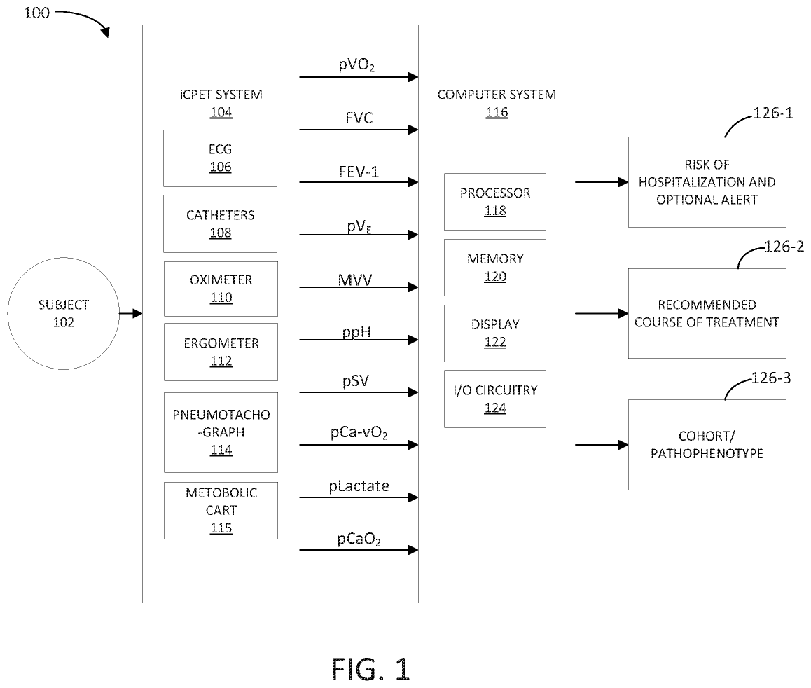

FIG. 1 shows an illustrative system 100 that includes a computer 102 and an iCPET system 104. The iCPET system 104 may be used to apply an iCPET test to a subject 102 in order to generate, in real-time, a variety of variables associated (at least) with oxygen and carbon dioxide expiration, ventilation, heart rate, blood chemistry, and blood pressure of the subject 102 before, during, and after exercise.

The iCPET system 104 may include a variety of devices and sensors including: an electrocardiogram (ECG) 106, catheters 108, an oximeter 110, an ergometer 112, a blood gas measurement device 113, a pneumotachograph 114, and a metabolic cart 115. It should be noted that the devices and sensors listed here are merely exemplary and, if desired, other applicable devices or sensors may be included in the iCPET system 104.

The ECG 106 may, for example, be a 12-lead ECG and may monitor heart rate and heart rhythm of the subject 102. The ECG 106 may measure ventricle volumes of the subject 102. From these measurements, peak stroke volume (pSV) of the subject 102, may be determined by subtracting the volume of the blood in a ventricle (e.g., the left ventricle) of the heart of the subject 102 at the end of a beat (end-systolic volume) from the volume of blood in the ventricle just prior to the beat (end-diastolic volume) at peak exercise.

The catheters 108 may include a radial artery catheter and/or a pulmonary artery catheter. The radial artery catheter may measure the blood pressure of the subject 102. The pulmonary artery catheter may measure the mean pulmonary artery pressure (mPAP) of the subject 102.

The oximeter 110 may be a pulse oximeter that indirectly monitors the oxygen saturation of the blood of the subject 102 and changes in the blood volume in the skin of the subject 102.

The ergometer 112 may be an upright cycle ergometer (e.g., a stationary bicycle). During an iCPET test, the subject may be seated on the ergometer 112 and may perform a predetermined amount (e.g., 3 minutes) of unloaded cycling at 55-65 rpm followed by a period of cycling with an incrementally increasing load (e.g., incrementally increasing at a rate of 5-30 Watts/min).

The blood gas measurement device 113 may be, for example, an arterial blood gas measurement device that may be used to measure peak arterial pH (ppH), peak arterial to mixed venous oxygen content difference (pCa-vO₂), peak arterial oxygen content (pCaO₂), and peak arterial lactate (pLactate) by analyzing the blood of the subject.

The pneumotachograph 114 may be integrated into a facemask worn by the subject being tested. The pneumotachograph may measure and record the rate of airflow corresponding to the breathing of the subject 102 during the administration of the iCPET test. The pneumotachograph 114 may also be used to measure forced vital capacity (FVC), forced expiratory volume in one second (FEV-1), peak minute ventilation (pV_{E}), and maximum voluntary ventilation (MVV) for the subject 102 during the administration of the iCPET test. While a pneumotachograph is described here, it should be noted that any other applicable spirometry measuring device may instead be used.

The metabolic cart 115 may be coupled (e.g., via tubing) to the facemask being worn by the subject 102 and may measure the amount of oxygen consumed and the amount of carbon dioxide produced by the subject 102 during the administration of the iCPET test, which may be used as a basis for calculating the peak rate (e.g., volume) of oxygen consumption (pVO₂) of the subject 102.

After administering an iCPET test to the subject 102, the values of variables generated by the iCPET system 104 (e.g., FVC, FEV-1, pVO₂, pV_{E}, MVV, ppH, pSV, pCa-vO₂, pCaO₂, and pLactate) may be transferred to the computer system 116.

The computer system 116 may include a processor 118, a memory 120, a display 122, and input/output (I/O) circuitry 124.

The processor 118 may be a computer processor that executes instructions that can be stored in the memory 120.

The memory 120 may include many types of non-transitory and/or transitory memory, data storage, or non-transitory computer-readable storage media, such as a first data storage for program instructions for execution by the processor 118, a separate storage for images or data, a removable memory for sharing information with other devices, etc.

The display 122 may be an electronic display that may include, for example, a light emitting diode (LED), liquid crystal display (LCD) screen, or any other applicable electronic screen.

The I/O circuitry 124 may include conventional inputs such as a keyboard, a mouse, a keypad, a microphone, or any other such device or element whereby a user can input a command to the computer system 116, and may include conventional outputs such as electronic speakers, printers, or any other such device or element whereby the computer system 116 may output physical representations of data.

The computer system 116, upon receiving the variable values from the iCPET system 104, may process the variable values (e.g., using processor 118) to classify the subject 102 as belonging to one of multiple cohorts, with each cohort corresponding to a respectively different pathophenotype of exercise dysfunction. This classification of the subject 102 may be performed by generating a vector that includes at least some of the variable values corresponding to the subject 102, and comparing the generated vector to multiple vector clusters corresponding to historical clinical data (e.g., clinical data collected, analyzed, and divided into vector clusters during preceding clinical tests). For example, the processor 118 may calculate the Euclidean distances between the generated vector and the centroid of each of the vector clusters, and may identify the vector cluster having the centroid with the shortest Euclidean distance from the generated vector. As will be described, each vector cluster may correspond to a respectively different cohort and associated pathophenotype of exercise dysfunction. The processor 118 may assign the subject 102 to the cohort and associated pathophenotype corresponding to the identified vector cluster. In some embodiments, the vector clusters (or just the centroids of the vector clusters) may be stored in the memory 120 of the computer system 116.

Once the subject 102 has been assigned to a cohort, the computer system 116 may generate and display a variety of outputs 126 containing prognostic information related to the subject 102 based on the cohort to which the subject 102 has been assigned. The output 126-1 includes a risk of hospitalization of the subject 102 determined based on the cohort of the subject 102, and optionally includes an alert indicating that the subject 102 should be hospitalized as soon as possible due to the severity of their exercise dysfunction.

The output 126-2 includes a recommended course of treatment for the subject 102 that is determined based on the cohort of the subject 102. For example, the recommended course of treatment may include the initiation of pharmacotherapeutic interventions such as treatment with a predetermined class of cardiovascular drugs that can be classified to ten categories based on the features, including, but not limited to peripheral vasodilator reaming enhancer, angiotensin converting agent inhibitor, hypotension and shock therapeutic agent, diuretic, antiarrhythmic agent, antiarralciton drug, antihypertensive agent, anticoagulant and thrombolytic agent, cardiac tonic therapeutic agents.

Peripheral vasodilator reaming enhancers, which can directly or indirectly effect on peripheral blood vessels to increase blood flow, include: Cilnidipine, Minoxidil, Prazosin HCl, Sildenafil citrate, Tadalafil (Adcirca), Nicorandil (Ikorel), Lacidipine (Lacipil, Motens), Benidipine hydrochloride, Cilazapril monohydrate (Inhibace), Fosinopril sodium (Monopril), Almotriptan malate (Axert), Milrinone (Primacor), Avanafil, Lomerizine HCl, Histamine Phosphate, Chromocarb and Pinacidil.

Angiotensin converting agent inhibitors, which are used to inhibit ACE activity and reduce the production of vasopressin II, so as to reduce the bradykinin hydrolysis, lead to vasodilatation, blood volume, and decrease blood pressure, include: Benazepril hydrochloride, Losartan potassium, Perindopril Erbumine (Aceon), Irbesartan (Avapro), Candesartan (Atacand), Olmesartan medoxomil (Benicar), Enalaprilat dehydrate, Telmisartan (Micardis), Ramipril (Altace), Valsartan (Diovan), Enalapril maleate (Vasotec), Candesartan cilexetil (Atacand), Conivaptan HCl (Vaprisol), Azilsartan Medoxomil (TAK-491) and Eprosartan Mesylate.

Hypotension and shock therapeutic agents, which include: L-Adrenaline (Epinephrine), DL-Adrenaline and Methoxamine HCl.

Diuretics, which are used to increase the generation of urine and increase the excretion of human body water, include: Bumetanide, Furosemide (Lasix), Metolazone (Zaroxolyn), Silodosin (Rapaflo), Chlorothiazide, Trichlormethiazide (Achletin), Torsemide (Demadex), Hydrochlorothiazide, Indapamide (Lozol), Dichlorphenamide (Diclofenamide), Amiloride hydrochloride dehydrate, Solifenacin succinate, Methyclothiazide, Benzthiazide, Meticrane, Bendroflumethiazide and Potassium Canrenoate.

Antiarrhythmic agents, which are used to inhibit abnormal heartbeat rhythm (arrhythmia), such as atrial fibrillation, atrial flutter, and ventricular tachycardia (ventricular tachycardia) and ventricular fibrillation, include: Adenosine (Adenocard), Dofetilide (Tikosyn), Amiodarone HCl, Ibutilide fumarate, Propafenone (Rytmonorm) and Disopyramide Phosphate.

Antiarralciton drugs, which are used to treat ischemic heart disease symptoms, include: Dexrazoxane Hydrochloride, Ranolazine dihydrochloride, Nisoldipine (Sular), Ranolazine (Ranexa), Acadesine, Nifedipine (Adalat), Amlodipine besylate (Norvasc), Diltiazem HCl (Tiazac), Ticagrelor and Oxprenolol HCl.

Antihypertensive agents are a medicament for hypertension treatment, which is used to prevent high blood pressure complications, such as stroke and myocardial infarction, and include: Bisoprolol, Doxazosin mesylate, Alfuzosin hydrochloride (Uroxatral), Nebivolol (Bystolic), Reserpine, Methyldopa (Aldomet), Eplerenon, Nimodipine (Nimotop), Betaxolol hydrochloride (Betoptic), Carvedilol, Metoprolol tartrate, Felodipine (Plendil), Amlodipine (Norvasc), Phentolamine mesilate, Imidapril (Tanatril) HCl, Aliskiren hemifumarate, Sodium Nitroprusside, Propranolol HCl, Levobetaxolol HCl, Esmolol HCl, (R)-(+)-Atenolol, Guanethidine Sulfate and Lofexidine HCl.

Anticoagulant, thrombolytic agents are the flag used to prevent blood coagulation (coagulation). Anticoagulants can treat thrombotic diseases, and include: Prasugrel (Effient), Cilostazol, Nafamostat mesylate, Clopidogrel (Plavix), Apixaban, Aminocaproic acid (Amicar), Dipyridamole (Persantine), Phenindione (Rectadione), Ticlopidine HCl, Ozagrel HCl, Argatroban, Bexarotene, Gabexate mesylate and Ozagrel and Anisindione.

Cardiac tonics may include beta blockers and calcium channel blockers. Cardiac tonics include: Pimobendan (Vetmedin), Ampiroxicam, Digoxigenin and Pindolol.

As another example, the recommended course of treatment may include optimization of bronchodilator therapy, pulmonary arterial hypertension therapy, inhaled corticosteroid therapy, muscarinic agents, immunomodulating agents, and pulmonary vasodilator therapy. As another example, the recommended course of treatment may include the initiation of non-pharmacotherapeutic intervention, including lifestyle changes such as prescription exercise, withdrawal of certain medicines and supplements, nutritional advice, and any other applicable lifestyle change appropriate given the cohort of the subject 102.

The output 126-3 may include the cohort and associated pathophenotype that was identified for the subject 102.

Based on the outputs 126 generated by the computer system 116, a physician may be able to accurately assess the severity of exercise dysfunction of the subject 102 and may make informed decisions regarding the appropriate treatment of the subject 102.

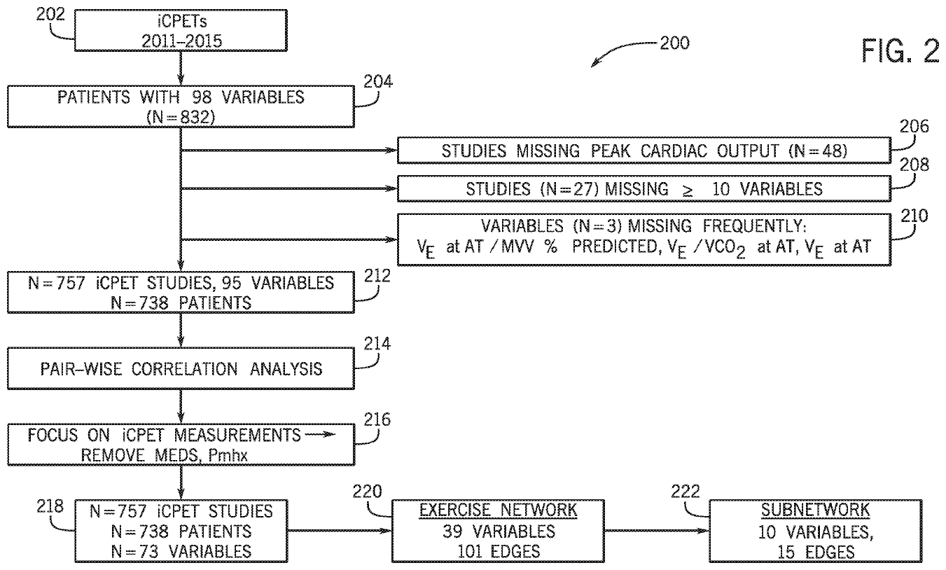

The vector clusters used as the basis for assigning the subject 102 to a particular cohort may be generated preceding the application of the iCPET test to the subject 102 using historical clinical data collected from multiple subjects to which iCPET tests have been applied. As will be described, this historical clinical data may be used not only to generate the vector clusters, but also to identify a subnetwork of interconnected variables that may be used to populate the vectors of the vector clusters. FIG. 2 shows an illustrative progression by which a subnetwork 222 may be derived from data collected during multiple clinical studies 202 (e.g., iCPET clinical studies in the present example), which may be conducted over the course of several years (e.g., from 2011 to 2015 in the present example). Clinical data 204 may be collected during the clinical studies 202. The clinical data 204 includes, for each subject of a number of subjects, values of multiple variables corresponding to that subject. In the present example, clinical data was collected for 832 subjects, with a total of 98 distinct variables being represented in the clinical data.

Significant numbers of variables may be missing in the clinical data 204 for some subjects of the clinical studies 202. Thus, clinical data from studies 206 and 208 may be omitted from the clinical data 204 to produce refined clinical data 212. In the present example, clinical data for the 48 subjects of the studies 206 was omitted from the refined clinical data 212, as this omitted clinical data lacked values for the variable corresponding to peak cardiac output. In the present example, clinical data for the 27 subjects of the studies 208 was omitted from the refined clinical data 212, as this omitted clinical data lacked values for more than 10 of the variables identified across all of clinical data 204.

Additionally, some of the variables identified across all of clinical data 204 may be found to be frequently missing from the clinical data of the subjects of the clinical studies 202, and may therefore be excluded from the refined clinical data 212. In the present example, three frequently missing variables 210 are omitted from the refined clinical data 212, including: minute ventilation during exercise at anaerobic threshold relative to maximum voluntary ventilation expressed as % predicted (V_{E} at AT/MVV % predicted), minute ventilation during exercise relative to carbon dioxide production at anaerobic threshold (V_{E}/VCO₂ at AT), and minute ventilation during exercise at anaerobic threshold (V_{E} at AT).

Finally, any missing variable values or variable values exceeding the variable mean by 5 standard deviations may be replaced with the variable mean value for that variable when generating the refined clinical data 212.

Once the refined clinical data 212 has been generated, pair-wise correlation analysis 214 may be applied to the refined clinical data 212. In the present example, 4,465 pair-wise correlations are observed from the 95 remaining variables, among which 1,061 are significant at the threshold P<10⁻¹⁰ and a correlation threshold of |r|>0.2 and included 92 of the 95 variables. The pair-wise analysis 214 generates a densely-connected network with low modularity (i.e., there is minimal separation among potential groups within the network) that may not be amenable to further analyses. Thus, variable removal 216 is performed on this densely-connected network in order to remove less relevant variables from the clinical data to generate further refined clinical data 218. In the present example, the variable removal 216 removes variables related to medical history and medication in order to focus the analysis on parameters obtained from iCPET testing, decreasing the number of variables in the clinical data to 73 to generate the further refined clinical data 218.

In order to increase the likelihood of capturing unexpected relationships between variables, the variables of the further refined clinical data 218 may be grouped such that variables with similar function are placed into the same functional group. The connections (e.g., correlations) between variables within the same functional group may then be removed and the correlation threshold may be raised to |r|>0.5. In the present example, the variables are categorized into the following groups: pulmonary function, exercise capacity, ventilatory response to exercise, oxygen transport and utilization, non-invasive cardiac performance, invasive cardiac performance, and systemic and cardiopulmonary hemodynamics.

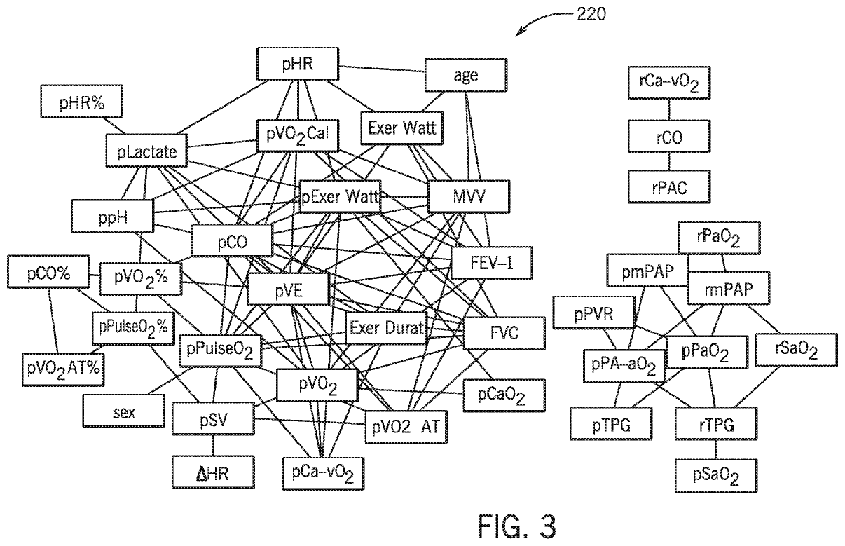

Any variables that remain unconnected after these changes may be removed from the clinical data to generate an exercise network 220, which is shown in FIG. 3. In the present example, the exercise network 220 may include 39 variables and 101 connections (e.g., edges). A principal component (PC) analysis may be performed to confirm that the top variables (e.g., the top 25 variables) contributing to the PC1 and PC2 of the PC analysis are present in the exercise network 220.

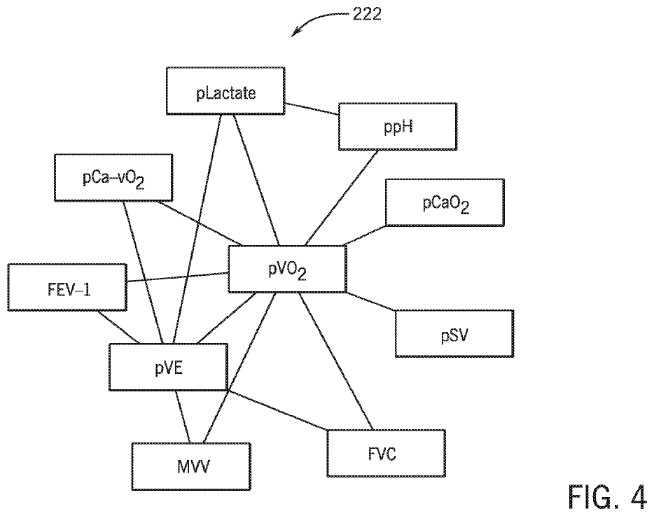

The exercise network 220 may be further refined to generate a smaller subnetwork 222, shown in FIG. 4, having a size amenable to additional analyses. In the present example, the subnetwork 222 may include the variable pVO₂ and all variables correlated to pVO₂, resulting in the subnetwork 222 including 10 variables and 15 connections. As shown, the subnetwork 222 includes the following variables: pVO₂, FVC, FEV-1, pV_{E}, MVV, ppH, pSV, pCa-vO₂, pCaO₂, and pLactate.

K-means clustering may be used to determine if unique groups (e.g., clusters) of subjects represented in the further refined clinical data 218 are identifiable based on the subnetwork 222. Table 1 shows relevant data corresponding to each of the four clusters identified via K-means clustering in the present example.

TABLE 1

| | | | | |
|---|---|---|---|---|
| Cluster 4 | Cluster 3 | Cluster 2 | Cluster 1 | |
| (N = 205) | (N = 260) | (N = l73) | [symbol] | P Value |

Clinical Characteristic

| | | | | | | | | | |
|---|---|---|---|---|---|---|---|---|---|
| Age (yr) | 70 | [61-76] | 58 | [49-68] | 50 | [36-62] | 48 | [35- | <0.0001 |
| Female (n, %) | 159 | (78) | 195 | (75) | 105 | (61) | 14 | (14) | <0.0001 |
| Weight (kg) | 80 | [68-95] | 73 | [64-91] | 77 | [64-95] | 92 | [78- | <0.0001 |
| BMI (kg/m2) | 29.3 | [24.3-34.4] | 26.8 | [23.1-31.6] | 26.0 | [23.0-31.5] | 27.4 | [24.4-31.1] | 0.001 |
| Age (yr) | 70 | [61-76] | 58 | [49-68] | 50 | [36-62] | 48 | [35- | <0.0001 |
| LVEF *(%) | 61.2 | [56.2-66.3] | 62.8 | [56.6-68.8] | 62.9 | [56.3-68.9] | 61.2 | [57.5-65.6] | 0.39 |

Co-morbidities

| | | | | | | | | | |
|---|---|---|---|---|---|---|---|---|---|
| Systemic hypertension | 132 | (64) | 100 | (38) | 54 | (31) | 28 | (28) | <0.0001 |
| Hyperlipidemia | 103 | (50) | 90 | (35) | 45 | (26) | 24 | (24) | <0.0001 |
| Diabetes mellitus | 52 | (25) | 25 | (10) | 13 | (8) | 4 | (4) | <0.0001 |
| ≥1 CHD risk factor | 36 | (18) | 47 | (18) | 25 | (14) | 15 | (15) | 0.7429 |
| Valvular disease | 27 | (13) | 16 | (6) | 5 | (3) | 3 | (3) | <0.0001 |
| History of tobacco | 5 | (2) | 6 | (2) | 5 | (3) | 3 | (3) | 0.8485 |
| Coronary artery disease | 32 | (16) | 23 | (9) | 16 | (9) | 3 | (3) | 0.0039 |

Medication Use

| Digoxin | 10 | (5) | 5 | (2) | 1 | (1) | 0 | | 0.0135 |
|---|---|---|---|---|---|---|---|---|---|
| β-adrenergic receptor | 80 | (39) | 69 | (27) | 45 | (26) | 10 | (10) | <0.0001 |
| antagonist | | | | | | | | | |
| Calcium channel antagonist | 54 | (26) | 37 | (14) | 11 | (6) | 6 | (6) | <0.0001 |
| ACE Inhibitor | 39 | (19) | 42 | (16) | 20 | (12) | 13 | (13) | 0.2131 |
| Diuretic | 90 | (44) | 60 | (23) | 28 | (16) | 13 | (13) | <0.0001 |
| Aspirin | 82 | (40) | 73 | (28) | 39 | (23) | 19 | (19) | <0.0001 |
| Insulin | 17 | (8) | 8 | (3) | 4 | (2) | 3 | (3) | 0.0229 |
| Oral hypoglycemic | 35 | (17) | 14 | (5) | 7 | (4) | 4 | (4) | <0.0001 |

Exercise subnetwork variables

| pVO2 (mL/kg/min) | 10.5 | [8.8-12.5] | 5.1 | [12.5-17.9] | 19.7 | [16.6-24.1] | 24.8 | [19.4-31.4] | <0.0001 |
|---|---|---|---|---|---|---|---|---|---|
| pVE (L) | 34 | [28-41] | 45 | [39-54] | 61 | [55-70] | 87 | [75- | <0.0001 |
| FEV-1 (% | 68 ± 21 | | 85 ± 19 | | 92 ± 18 | | 97 ± 16 | | <0.0001 |
| FVC (% predicted) | 67 ± 19 | | 87 ± 17 | | 93 ± 16 | | 98 ± 16 | | <0.0001 |
| MVV(L) | 57 | [44-69] | 85 | [71-96] | 104 | [92-117] | 132 | [117- | <0.0001 |
| ppH | 7.40 | [7.38-7.45] | 7.39 | [7.36- | 7.37 | [7.34-7.39] | 7.36 | [7.32- | <0.0001 |
| pLactate (mg/dL) | 3.4 | [2.4-4.5] | 4.9 | [3.9-5.9] | 6.6 | [5.3-7.8] | 7.0 | [5.3- | <0.0001 |
| pCaO₂ (mL/dL) | 16.2 ± 1.9 | | 18.5 ± 1.8 | | 19.2 ± 1.7 | | 21.1± | | <0.0001 |
| pCa—VO₂ (mL/dL) | 10.1 | [8.8-11.0] | 11.5 | [10.2- | 12.0 | [11.0-13.5] | 14.2 | [12.2- | <0.0001 |
| pSV (mL) | 76.7 | [64.1-94.2] | 76.5 | [64.0-88.9] | 86.2 | [75.1-107.2] | 110.5 | [92.7-129.3] | <0.0001 |

[symbol]

indicates data missing or illegible when filed

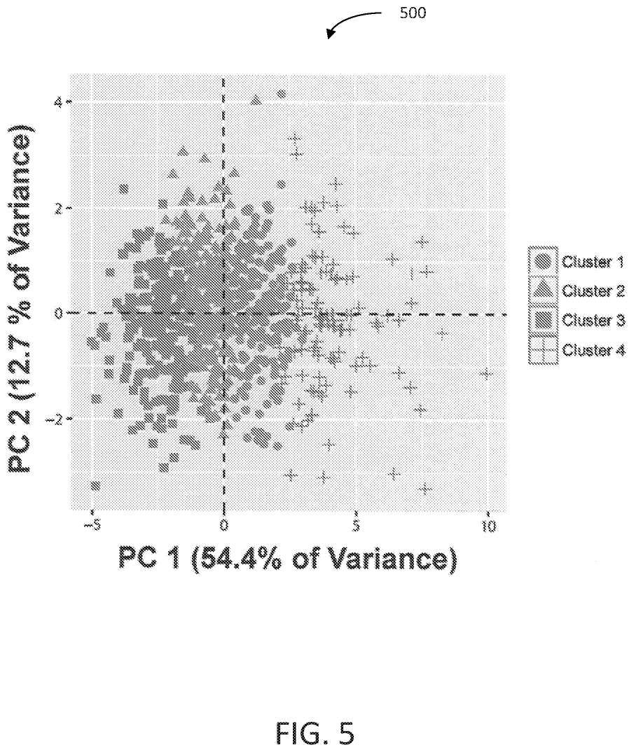

FIG. 5 shows chart 500 depicting a distribution of the 738 subjects of the present example in the four identified clusters plotted by PC1 and PC2 of a PC analysis. One purpose of this PC analysis is to verify the contribution of each variable to the overall variance of the population (e.g., all 738 subjects represented in the further refined clinical data 218). In the present example, all ten variables contributed to the first three prinicipal component vectors.

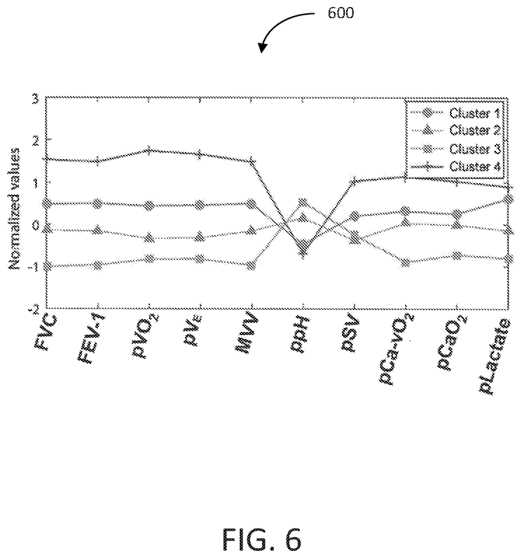

The values of each variable in subnetwork 222 for each of the 738 subjects of the present example may be normalized with a mean of 0 and variance of 1. FIG. 6 shows an illustrative chart 600 depicting the normalized values of clinical data for each variable in the subnetwork 222 stratified by cluster in accordance with an embodiment. Specifically, the lowest normalized values for 9 of the 10 subnetwork variables may be observed in cluster 3, with incremental increases observed in cluster 2, cluster 1, and finally cluster 4. For arterial pH at peak exercise (ppH), the trend is directionally opposite and the magnitude in difference across the clusters is less compared to that of the other 9 variables. The estimated 3-year all-cause hospitalization rates for clusters 3, 2, 1, and 4 of the present example is 42%, 34%, 17%, and 2%, respectively (P<0.0001).

For a given cluster defined by the k-means cluster analysis of the further refined clinical data 218 using the variables of the subnetwork 222, an associated vector cluster may be defined, with each vector cluster including multiple vectors, with each of the multiple vectors corresponding to a single subject of the clinical studies 202 and including values corresponding to that subject for each of the variables defined in the subnetwork 222. In order to distinguish between the subject clusters described above and the vector clusters now described, the subject clusters identified through k-means analysis may be referred to as “cohorts,” as mentioned previously. Each vector cluster may have a defined centroid (e.g., a multi-dimensional average). The centroid of each vector cluster may be used in subsequently assigning a subject to a cohort corresponding to one of the vector clusters. For example, Euclidean distances between a vector of variable values corresponding to a given subject and the centroids of each of the vector clusters may be calculated (e.g., by the processor 118 of the computer system 116 of FIG. 1) and the subject may be assigned to the cohort corresponding to the vector cluster corresponding to the shortest of these Euclidean distances.

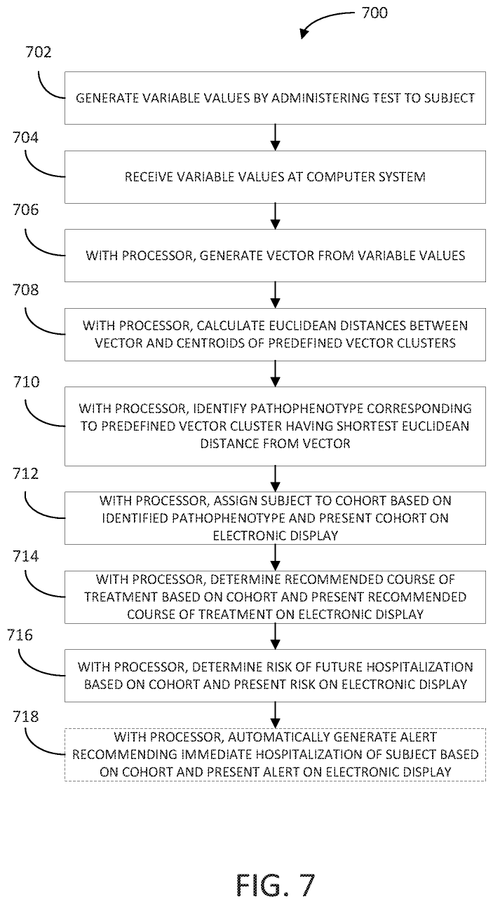

FIG. 7 shows an illustrative flow chart for a method 700 by which a system (e.g., system 100 of FIG. 1) may automatically generate and display prognostic information based on variables generated by administering a test (e.g., administering an iCPET test with the iCPET system 104 of FIG. 1) to a subject (e.g., subject 102 of FIG. 1) in accordance with an embodiment. The method 700 may be performed, at least in part, by using a processor to execute instructions stored in the memory of a computer system (e.g., memory 120 of computer system 116 of FIG. 1).

At step 702, variable values are generated by administering a test to the subject. For example, an iCPET system may be used to apply an iCPET test to a subject to generate iCPET data that includes values for variables including: pVO₂, FVC, FEV-1, pV_{E}, MVV, ppH, pSV, pCa-vO₂, pCaO₂, and pLactate.

At step 704, a computer system receives the variable values generated via the administration of the test. For example, the computer system may receive the variable values over a direct connection to an iCPET system, or may receive the variables over an electronic communications network such as the internet.

At step 706, a processor of the computer system generates (e.g., by executing instructions stored in a memory of the computer system) a vector containing the variable values. In some embodiments, each of the variable values may be normalized to have an updated mean of zero and an updated variance of one relative to a respective mean and a respective variance of values for that variable represented in historical clinical data for multiple subjects (e.g., represented in the predefined vector clusters described below) before being added to the generated vector.

At step 708, the processor of the computer system calculates Euclidean distances between the vector and respective centroids of multiple predefined vector clusters. Each of these predefined vector clusters may, for example, correspond to a respectively different cohort and pathophenotype of a disease and condition and may be predefined based on analysis (e.g., k-means clustering) of the historical clinical data for the multiple subjects.

At step 710, the processor of the computer system identifies the pathophenotype corresponding to the predefined vector cluster corresponding to the shortest of the calculated Euclidean distances. For example, the processor may identify the pathophenotype according to a look-up-table (LUT) or database in the memory of the computer system that defines relationships between the predefined vector clusters and various pathophenotypes.

At step 712, the processor may assign the subject to a cohort based on the identified pathophenotype (e.g., according to a LUT or database in memory) and may present the cohort on an electronic display of the computer system (e.g., display 122 of FIG. 1). Optionally, the identified pathophenotype may also be presented using the electronic display at this step.

At step 714, the processor may determine a recommended course of treatment based on the cohort to which the subject has been assigned and may present this recommended course of treatment on the electronic display. Examples have recommended courses of treatment for exercise dysfunction have been described previously.

At step 716, the processor may determine a risk of future hospitalization based on the cohort to which the subject has been assigned and may present this risk on the electronic display.

At step 718, optionally, the processor may automatically generate an alert recommending immediate hospitalization of the subject based on the cohort to which the subject has been assigned and may present this alert on the electronic display. For example, this alert may only be generated for one or more predefined cohorts corresponding to an extremely high (e.g., above a defined threshold) risk of near-term hospitalization. In some embodiments, this alert may include other outcomes including, but not limited to, risk of mortality of the subject, pharmacotherapeutic initiation, and pharmacotherapeutic escalation. It should be noted that such high-risk cohorts may not exist for some diseases or disorders.

One non-limiting example of a clinical application of one embodiment of the present invention will now be described. A 67-year old male patient presents to the cardiology office with a complaint of exertional breathlessness. The symptoms have been progressive for 2 years, and, currently he reports symptoms consistent with New York Heart Association Functional Class II. Baseline electocardiography for the patient is normal and resting echocardiography shows normal left ventricluar function with mildly enlarged left atrium. A vasodilator nuclear perfusion imaging study is performed to assess coronary artery disease, but shows no evidence of myocardial ischemia. The patient is referred for iCPET testing. The results of the iCPET test demonstrate a modest elevation in pulmonary artery wedge pressure with exercise, suggesting that diuretic therapy is indicated. With conventional diagnostic systems, this would likely be the only information provided to the referring physician. However, using a method (e.g., in conjunction with a computer system such as computer system 116 of FIG. 1) for generating prognostic information corresponding to that described above (e.g., the method of FIG. 7), abnormalities in pulmonary function, skeletal muscle oxygen uptake, and right ventricular systolic function are identified in the patient. The method also generates an estimate of a 33% chance for hospitalization over the following 3-years for the patient, identifying the patient as being at high clinical risk. Outcomes generated by the application of the method result in review of the patient's medical and clinical profile, which would not have otherwise occurred. Based on the information generated via the application of the method, the referring physician makes the following recommendations to the patient: i) the addition of bronchodilator therapy, ii) a decrease in beta-receptor adrenergic therapy dose, iii) prescription exercise, iv) 10% weight loss, v) evaluation by an obstructive sleep apnea specialist, and vi) consideration to future therapy with pulmonary vasodilator treatments.

1. A method comprising:
receiving, with a computer system, values of one or more variables corresponding to a subject to which a diagnostic test has been administered, wherein the diagnostic test comprises invasive cardiopulmonary exercise testing (iCPET);
with a processor of the computer system, generating at least one vector from the values of the one or more variables;
with the processor, determining a plurality of Euclidean distances between the at least one vector and respective centroids of each of a plurality of predefined vector clusters corresponding to a plurality of pathophenotypes;
with the processor, identifying a pathophenotype corresponding to the predefined vector cluster of the plurality of predefined vector clusters corresponding to the shortest Euclidean distance of the plurality of Euclidean distances;
with the processor, assigning the subject to a cohort based on the identified pathophenotype;
with the processor, determining a recommended course of treatment based on the cohort to which the subject has been assigned; and
presenting the recommended course of treatment on an electronic display of the computer system.

2. (canceled)

3. The method of claim 1, wherein the one or more variables include at least one of, but are not limited to: peak minute ventilation, forced expiratory volume in one second, peak stroke volume, maximum voluntary ventilation, forced vital capacity, peak arterial to mixed venous oxygen content difference, peak arterial pH, peak arterial lactate, peak arterial oxygen content, and peak rate of oxygen consumption.

4. The method of claim 1, wherein the recommended course of treatment includes initiation of pharmacotherapeutic intervention including treatment with a predetermined class of cardiovascular drugs that can be classified to ten categories based on the features, including, but not limited to pulmonary vasodilator therapy, pulmonary arterial hypertension treatment, peripheral vasodilator reaming enhancer, angiotensin converting agent inhibitor, hypotension and shock therapeutic agent, diuretic, antiarrhythmic agent, antiarralciton drug, antihypertensive agent, anticoagulant and thrombolytic agent, and cardiac tonic therapeutic agents including, but not limited to beta blockers and calcium channel blockers.

5. The method of claim 1, wherein the recommended course of treatment includes optimization of bronchodilator therapy, inhaled corticosteroid therapy, muscarinic agents, or immunomodulating agents.

6. The method of claim 1, further comprising:
with the processor, automatically generating an alert based on the cohort to which the subject has been assigned, wherein the alert recommends outcomes including, but not limited to immediate hospitalization of the subject, risk of mortality of the subject, pharmacotherapeutic initiation, and pharmacotherapeutic escalation; and
presenting the alert on the electronic display.

7. The method of claim 1, wherein the plurality of predefined vector clusters comprises additional values for each of the one or more variables, the method further comprising:
with the processor, normalizing each of the values of the one or more variables corresponding to the subject relative to a respective mean and a respective variance of corresponding values of the additional values to have an updated mean of zero and an updated variance of one before generating the at least one vector.

8. A method comprising:
administering, with an invasive cardiopulmonary exercise testing (iCPET) system, an iCPET test to a subject;
during the administration of the iCPET test, continuously collecting and storing iCPET data captured in real-time by the iCPET system;
with a computer processor, analyzing values of one or more variables of the iCPET data using network analysis to identify an exercise pathophenotype of the subject;
with the computer processor, assigning the subject to a cohort based on the identified exercise pathophenotype;
with the computer processor, determining a recommended course of treatment based on the cohort to which the subject has been assigned; and
presenting the recommended course of treatment on an electronic display.

9. The method of claim 8, wherein analyzing the values of the one or more variables of the iCPET data using network analysis to identify an exercise pathophenotype of the subject further comprises:
with the computer processor, generating at least one vector from the values of the one or more variables;
with the computer processor, determining a plurality of Euclidean distances between the at least one vector and respective centroids of each of a plurality of predefined vector clusters corresponding to a plurality of exercise pathophenotypes; and
with the computer processor, identifying the exercise pathophenotype as that which corresponds to the predefined vector cluster of the plurality of predefined vector clusters corresponding to the shortest Euclidean distance of the plurality of Euclidean distances.

10. The method of claim 8, wherein the one or more variables include at least one of, but are not limited to: peak minute ventilation, forced expiratory volume in one second, peak stroke volume, maximum voluntary ventilation, forced vital capacity, peak arterial to mixed venous oxygen content difference, peak arterial pH, peak arterial lactate, peak arterial oxygen content, and peak rate of oxygen consumption.

11. The method of claim 8, wherein the one or more variables include peak minute ventilation, forced expiratory volume in one second, peak stroke volume, maximum voluntary ventilation, forced vital capacity, peak arterial to mixed venous oxygen content difference, peak arterial pH, peak arterial lactate, peak arterial oxygen content, and peak rate of oxygen consumption.

12. The method of claim 8, wherein the recommended course of treatment includes initiation of pharmacotherapeutic intervention including treatment with a predetermined class of cardiovascular drugs that can be classified to ten categories based on the features, including, but not limited to pulmonary vasodilator therapy, pulmonary arterial hypertension treatment, peripheral vasodilator reaming enhancer, angiotensin converting agent inhibitor, hypotension and shock therapeutic agent, diuretic, antiarrhythmic agent, antiarralciton drug, antihypertensive agent, anticoagulant and thrombolytic agent, and cardiac tonic therapeutic agents including, but not limited to beta blockers and calcium channel blockers.

13. The method of claim 8, wherein the recommended course of treatment includes optimization of bronchodilator therapy, inhaled corticosteroid therapy, muscarinic agents, or immunomodulating agents.

14. The method of claim 8, further comprising:
with the computer processor, automatically generating an alert based on the cohort to which the subject has been assigned, wherein the alert recommends immediate hospitalization of the subject; and
presenting the alert on the electronic display.

15. The method of claim 8, wherein analyzing the values of the one or more variables of the iCPET data using network analysis to identify an exercise pathophenotype of the subject further comprises:
with the computer processor, normalizing each of the values of the one or more variables to have an updated mean of zero and an updated variance of one relative to a respective mean and a respective variance of additional values for a corresponding variable of the one or more variables represented in the plurality of predefined vector clusters;
with the computer processor, generating a vector that includes the normalized values;
with the computer processor, determining a plurality of Euclidean distances between the vector and respective centroids of each of a plurality of predefined vector clusters; and
with the computer processor, identifying the exercise pathophenotype as that which corresponds to the predefined vector cluster of the plurality of predefined vector clusters corresponding to the shortest Euclidean distance of the plurality of Euclidean distances.

16. A system comprising:
an invasive cardiopulmonary exercise testing (iCPET) system that administers an iCPET study on a subject and that generates values for a plurality of variables for the subject during the administration of the iCPET study; and
a computer system that is communicatively coupled to the iCPET system, the computer system comprising:
a memory;
an electronic display; and
a processor that executes instructions stored in the memory for:
receiving, from the iCPET system, the values for the plurality of variables;
analyzing the values of the plurality of variables using network analysis to identify an exercise pathophenotype of the subject;
assigning the subject to a cohort based on the identified exercise pathophenotype;
determining a recommended course of treatment based on the cohort to which the subject has been assigned; and
presenting the recommended course of treatment on the electronic display, wherein the recommended course of treatment includes initiation of pharmacotherapeutic intervention including treatment with a predetermined class of cardiovascular drugs that can be classified to ten categories based on the features, including, but not limited to pulmonary vasodilator therapy, pulmonary arterial hypertension treatment, peripheral vasodilator reaming enhancer, angiotensin converting agent inhibitor, hypotension and shock therapeutic agent, diuretic, antiarrhythmic agent, antiarralciton drug, antihypertensive agent, anticoagulant and thrombolytic agent, and cardiac tonic therapeutic agents including, but not limited to beta blockers and calcium channel blockers.

17. The system of claim 16, wherein the plurality of variables includes at least one of, but is not limited to: peak minute ventilation, forced expiratory volume in one second, peak stroke volume, maximum voluntary ventilation, forced vital capacity, peak arterial to mixed venous oxygen content difference, peak arterial pH, peak arterial lactate, peak arterial oxygen content, and peak rate of oxygen consumption.

18. The system of claim 16, wherein each of the plurality of variables is correlated with at least one other variable of the plurality of variables with a correlation coefficient having a magnitude greater than 0.5 and a calculated probability of less than 10⁻⁴⁰.

19. (canceled)

20. The system of claim 16, wherein the recommended course of treatment includes initiation of pulmonary vasodilator therapy, inhaled corticosteroid therapy, muscarinic agents, or immunomodulating agents.

21. The system of claim 16, wherein the processor further executes instructions for:
automatically generating an alert based on the cohort to which the subject has been assigned, wherein the alert recommends immediate hospitalization of the subject; and
presenting the alert on the electronic display.

22. The method of claim 16, wherein the processor further executes instructions for:
normalizing each of the values of the plurality of variables to have an updated mean of zero and an updated variance of one relative to a respective mean and a respective variance of additional values for a corresponding variable of the plurality of variables represented in the plurality of predefined vector clusters;
generating a vector that includes the normalized values;
determining a plurality of Euclidean distances between the vector and respective centroids of each of a plurality of predefined vector clusters; and
identifying the exercise pathophenotype as that which corresponds to the predefined vector cluster of the plurality of predefined vector clusters corresponding to the shortest Euclidean distance of the plurality of Euclidean distances.

23. (canceled)
